(19) United States
(12) Patent Application Publication
(10) Pub. No.: US 2009/0232849 A1
(43) Pub. Date: Sep. 17, 2009

(54) METHODS AND COMPOSITIONS FOR THE TREATMENT OF CANCER

(75) Inventors: Bernard Gallez, Lillois (BE); Reginald Ansiaux, Namur (BE)

Correspondence Address: KNOBBE MARTENS OLSON & BEAR LLP, 2040 MAIN STREET, FOURTEENTH FLOOR, IRVINE, CA, 92614, US

(73) Assignee: Universite Catholique de Louvain, Louvain-La-Neuve (BE)

(21) Appl. No.: 11/817,223

(22) PCT Filed: Oct. 17, 2005
(86) PCT No.: PCT/EP2005/011145

§ 371 Date: Oct. 31, 2008

(30) Foreign Application Priority Data
Mar. 3, 2005 (EP) 05447045.5

(51) Int. Cl. A61K 39/08; C12N 5/02; C12N 13/00; A61K 39/02

(52) U.S. Cl. 424/239.1; 435/375; 435/173.1; 424/236.1

(57) ABSTRACT

Compositions and a method for sensitizing a cancer for treatment by cytotoxic therapy is disclosed. The method includes the step of administering to the cancerous tissue or cells a pharmaceutical composition containing a Botulinum toxin, BT.

### BACKGROUND TO THE INVENTION

Cancer is one of the major causes of adult death, but despite extensive investment in research, treatment and early diagnosis, more than half of patients diagnosed with cancer die within 5 to 7 years. Widely used cancer treatments include surgical operations, radiotherapy, chemotherapy, and combinations thereof.

Among said treatments, surgical operations treat localised growths, having limited effects only on the sites where the cancerous cells or tumours are excised. Accordingly, surgical operations are not effective in cases where it is impossible to excise cancerous cells or tumours due to their inaccessible location, or in cases where cancerous cells or tumours already spread from an original site to one or more sites (especially, including important organs) in the patient's body.

Radiotherapy is also a local cancer treatment generally having effects on targeted irradiation sites. It works in some types of cancers, but this is not always dependent on the localisation. Radiotherapy has been reported to be effective in treating specific kinds of cancers such as prostate cancer and head and neck tumours.

With regard to chemotherapy, such treatment is usually not localized, rather the anticancer agents are administered and are effective systemically. Targeted cancerous cells or tumours generally develop resistance at an early stage to chemotherapy or during chemotherapy. Thus a number of chemotherapeutic drugs, which have been developed and approved can be ineffective in treating cancers at later stages of therapy.

Consequently, in order to enhance anticancer treatment, there is a need to develop effective chemotherapy, radiotherapy treatment and/or combination thereof, especially for cases where surgical operations are difficult and the risk of recurrence is high.

Known in the art are chemotherapy-radiotherapy combinations. However, such combination cancer treatments have been proved to be unsatisfactory in many circumstances. Among anticancer drugs developed to date, it has been reported that taxol and cisplatin may be used as radiotherapy-enhancing agents (or so called “radio-sensitiser”). However, even though they are rather effective in enhancing radiotherapy, they still have some critical defects, especially causing toxicity to patients.

In view of the disadvantages of the art, there is a need for a cancer treatment which can enhance the effectiveness of existing therapies such as radiotherapy and chemotherapy, while having a low toxicity effect.

### BRIEF DESCRIPTION OF THE FIGURES

FIG. 1 Effect of a single intratumoral injection of BTTA on FSaII tumour (A) and TLT tumour (B) oxygenation monitored by EPR Oximetry. ▴ control group (n=8 and 4 for FSAII and TLT respectively). ◯, treated group (n=10 and 4 for FSAII and TLT respectively). Note the significant increase in pO₂ 24 hours after the injection with a maximum on day 3. Points, mean; bars, SE; **, P<0.01.

FIG. 2 a) Typical MRI images of FSaII tumors showing the perfused pixels 3 days after treatment or vehicle. b) Mean percentage of perfused pixels for treated (n=3) and control group (n=4). c) Distribution of pharmacokinetics parameters in tumours treated with vehicle or BTTA. Vp is the blood plasma volume per unit volume of tissue, K^{trans} is the influx volume transfer constant from plasma into the interstitial space, Kₑₚ is the efflux volume transfer constant from the interstitial space back to the plasma. d) Overall estimation of pharmacokinetic parameters after BoNT-A treatment. Columns, mean; bars, SE. **, P<0.01; ns, not significant.

FIG. 3 a) Typical images of FSAII tumours stained by the Patent Blue three days after the injection of vehicle or BTTA. Note that the FSAII treated tumor stained more positive than the control. b) Effect of BTTA on tumor perfusion assessed by Patent Blue Staining three days after the single injection. Columns, mean value of tumor percentage of coloured area for control group (n=5) and treated group (n=8). ***, P<0.001.

FIG. 4 Uptake of Albumine-Evans Blue in FSaII tumours. A non significant increase is observed for treated groups (8.30±1.38 (n=4) vs 4.38±0.92 μg/g FSaII tumour (n=4) for treated and control group respectively). Columns, mean value of Evans Blue uptake (μg/gtumor). ns, not significant

FIG. 5 Effect of the combination of BTTA and radiation on FSaII tumour regrowth. Mice were treated with BTTA (open triangles; n=6), treated with vehicle (open squares; n=5), treated with 20 Gy of RX three days after the injection of the vehicle (solid squares; n=5), or treated with 20 Gy of RX three days after the injection of BTTA (solid triangles; n=6). Each point represents the mean tumour size±SE. Day 0 corresponds to the irradiation day. No difference in regrowth delay was observed between the control and treated groups alone. Regrowth delays to double tumour diameter were 11.04±0.21 days for control+RX and 15.70±1.03 days for BTTA+RX (P<0.01). BoTN-A increased the regrowth delay by a factor 2.3.

FIG. 6 Effect of BTTA (0.73 u/100.10⁶ cells) on FSaII cells evaluated by the Trypan Blue Exclusion Dye Method (a) and by the Clonogenic Cell Survival Assay (b). Both techniques showed an effect of the irradiation of 2 Gy on tumor cells. Nevertheless, BTTA did not exert any sensitising effect regardless of the methods used. *, P<0.05; **, P<0.01; ns: not significant.

FIG. 7 Effect of the combination of BTTA and chemotherapy on TLT tumor regrowth. Mice were treated with BTTA (open triangles; n=8), treated with vehicle (open squares; n=8), treated with cyclophosphamide three days after the injection of the vehicle (stars; n=7), or treated with cyclophosphamide three days after the injection of BTTA (solid triangles; n=6). Each point represents the mean tumour size±SE. The injection of BTTA or vehicle was performed on day 0. No difference in regrowth delay was observed between control, BTTA and vehicle+cyclophosphamide groups. Regrowth delays to double tumour diameter were 7.73±0.38 days for vehicle+cyclophosphamide and 11.68±0.46 days for BTTA+cyclophosphamide (P<0.001). BTTA increased the regrowth delay by a factor 5.1.

FIG. 8 Effect of BTTA on the caspase-3 activation. Values are presented as percentage of control. Note the increase in the activation for BTTA treated mice when combined treatment is applied (1.9 fold increase with radiotherapy (A) and 4.7 fold increase with chemotherapy (B)). Columns, % of control; bars, SE. *, P<0.05; ***, P<0.001.

FIG. 9 Effect of BTTA on the relaxation to phentolamine. Representative tracings showing the relaxation of preconstricted co-opted saphenous arteries to phentolamine in the presence (b) or the absence (a) of BTTA treatment.

FIG. 10 Bar graph of the concentration values for the control and BTTA treated tumours, and the corresponding signal of gemcitabine detected by NMR.

### DETAILED DESCRIPTION OF THE INVENTION

Unless defined otherwise, all technical and scientific terms used herein have the same meaning as is commonly understood by one of skill in the art. All publications referenced herein are incorporated by reference thereto.

The articles “a” and “an” are used herein to refer to one or to more than one, i.e. to at least one of the grammatical object of the article. By way of example, “a sample” means one sample or more than one sample.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of samples, and can also include 1.5, 2, 2.75 and 3.80, when referring to, for example, concentrations).

The present invention is based on the surprising finding by the inventors that tumours become sensitised to cytotoxic therapies when they are pre-treated with a botulinum toxin (BT).

A BT refers to any one of the naturally occurring botulinum toxins found in Clostridium botulinum species. To date, eight different BT types have been isolated and characterised from various strain of C. botulinum. The isolated toxins, distinguished largely by neutralisation with type-specific antibodies, have been accorded the names botulinum toxin type A (known as BTTA herein), B (BTTB), C1 (BTTC1), C2 (BTTC2), C3 (BTTC3), D (BTTD), E (BTTE), F (BTTF) and G (BTTG).

The pre-treatment may be with a single BT, or with two or more BTs together in a composition. Where there are two or more BTs, one BT may be administered simultaneously, separately or sequentially with respect to another BT.

One aspect of the invention is a pharmaceutical composition comprising two or more BTs for simultaneous, separate or sequential administration to a subject.

One aspect of the invention is a method for treating cancer comprising administering to an individual an effective amount of a composition comprising two or more BT, wherein said BTs are administered simultaneously, separately or sequentially.

By simultaneous administration means the BTs administered to a subject at the same time. For example, as a mixture or a composition comprising said components. An example is as a solution comprising the components of interest.

By separate administration means the BTs are administered to a subject at the same time or substantially the same time. The components may be present in a kit as separate, unmixed preparations. For example, the separate BTs may be present in the kit as individual vials. The inhibitors may be administered to the subject by separate injections at the same time, or injection directly following the other.

By sequential administration means at least two are administered to a subject sequentially. The individual BTs may be present in a kit as separate, unmixed preparations. There is a time interval between doses. For example, one component might be administered up to 336, 312, 288, 264, 240, 216, 192, 168, 144, 120, 96, 72, 48, 24, 20, 16, 12, 8, 4, 2, 1, 0.5, 0.4, 0.3, 0.2, or 0.1 hours after the other component.

In sequential administration, one component may be administered once, or any number of times and in various doses before and/or after administration of another component. Sequential administration may be combined with simultaneous or sequential administration.

The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than botulinum toxin type B. Additionally, botulinum toxin type B has been determined to be non-toxic in primates at a dose of 480 U/kg which is about 12 times the primate LD50 for botulinum toxin type A. One unit (U) of botulinum toxin is defined as the LD50 upon intraperitoneal injection into female Swiss Webster mice weighing 18-20 grams each. Botulinum toxin apparently binds with high affinity to cholinergic motor neurons, is translocated into the neuron and blocks the release of acetylcholine.

Clostridial neurotoxins are generally made by the bacterium as a single inactive polypeptide chain (150 kD) and released (cleaved) by bacterial cell lysis. Cleavage or “nicking” by endogenous proteases activates the toxin and yields the active dichain form of the toxin: light Chain (53 kD) and a heavy Chain (97 kD) joined by a single disulfide bond and noncovalent bonds. Botulinum toxins are generally purified from bacterial filtrates in complex with other, non-toxic proteins.

One embodiment of the present invention is a method for treating cancerous cells or tumours by cytotoxic therapy comprising the step of administering to said cells or tumours a pharmaceutical composition comprising one or more BTs.

Another embodiment of the present invention is a use of pharmaceutical composition comprising one or more BTs, for the manufacture of a medicament for the treatment cancerous cells or tumours in combination with cytotoxic therapy.

Another embodiment of the present invention is a pharmaceutical composition comprising one or more BTs for the treatment cancerous cells or tumours.

Another embodiment of the present invention is a method for enhancing radiotherapy treatment of cancerous cells or tumours comprising the step of administering to said cells or tumours one or more BTs.

Another embodiment of the present invention is a method for enhancing chemotherapy treatment of cancerous cells or tumours comprising the step of administering to said cells or tumours one or more BTs.

Another embodiment of the present invention is a method of sensitising cancerous cells or tumours to systemic radiotherapy, comprising administering to said cells or tumours one or more BTs.

Another embodiment of the present invention is a method of sensitising cancerous cells or tumours to systemic chemotherapy, comprising administering to said cells or tumours one or more BTs.

Where the embodiments herein describe a method using the present composition, they can equally be taken to mean a use of a composition, and vice versa.

Another embodiment of the present invention is a method of increasing the uptake of an active compound in cancerous cells or tumours, comprising administering to said cells or tumours one or more BTs. The active compound can be any agent useful in the treatment of cancer such as a cytotoxic agent, transcription or translation control substance, antisense compound, or other substances known to the skilled person.

In the above embodiments, preferably said composition is administered to said cells or tumours. Preferably one or more BTs is administered in an effective amount.

The above mentioned embodiments make use of one or more BTs; however, another aspect of the invention uses a nucleic acid capable of expressing a BT. Therefore, the above mentioned embodiments may apply where a BT is substituted for nucleic acid encoding a BT in a composition, use or method described herein. Where the BT is delivered as nucleic acid, said nucleic acid may be present in a vector which allows expression of the BT in situ. Alternatively, it may be present in a host strain such as a bacteria, phage, fungi, such delivery vehicles known to the person skilled in the art.

### Type A

A BTTA refers to all forms of BT comprising the Clostridium botulinum type A neurotoxin. BTTA is known to exist naturally in three forms each comprising the type A neurotoxin:
the M complex (300 kD) consisting of the neurotoxin polypeptide plus a non-toxic non-hemagglutinin protein of similar size;
the L complex (500 kD);
the LL complex (900 kD) which consists of a number of proteins with hemagglutinin activity in addition to the proteins in the M complex.

These above mentioned forms can fall within the meaning of a BTTA according to the present invention.

BTTA may include the commercial products Botox® (Botulinum Toxin Type A Neurotoxin Complex, Allergan), Botox® Cosmetic (Allergan), Vistabel® (Allergan, France), Dysport® (Ipsen Ltd./Beaufour Ipsen), Reloxin™ (Ipsen Ltd./Inamed), Clostridium botulinum type A toxins prepared by Mentor Corporation, Xeomin® (Merz Pharma, Germany), Linurase® (Prollenium, Inc., Canada), CBTX-A® (Lanzhou Biological Products Institute, China), and Neuronox® (Medy-Tox, Inc., South Korea). These above mentioned products are within the scope of a BTTA according to the present invention.

According to one aspect of the invention a BTTA polypeptide is Nc-224 (Allergan and CAMR), a fragment of botulinum toxin standard A lacking the binding domain (LHN/A), conjugated to native Erythrina crystagalli lectin (ECL). The lectin selectively targets the toxins to A-delta and C fibres (U.S. Pat. No. 4,734,275).

According to another aspect of the invention a BTTA polypeptide is Nc-270 (Allergan and CAMR), a fragment of botulinum toxin standard A lacking the binding domain (LHN/A), conjugated to recombinant Erythrina crystagalli lectin (ECL). The lectin selectively targets the toxins to A-delta and C fibres (U.S. Pat. No. 4,734,275).

According to another aspect of the invention a BTTA polypeptide is a highly purified form of botulinum neurotoxin standard A developed by Ipsen and by Inamed Corporation. It includes the commercial products Dysport® and Reloxin™.

BTTA may include a complex-free type-A neurotoxin. BTTA may also include a polypeptide comprising the sequence of the active BTTA neurotoxin polypeptide. It may also include a polypeptide with a similar identity, homology to, or comprising a functional fragment of the native, active type-A neurotoxin polypeptide.

Nucleic acid encoding BTTA may comprise a nucleic acid sequence capable of encoding the active type-A neurotoxin polypeptide. It may alternatively comprise a nucleic acid sequence capable of encoding a polypeptide with a similar identity, homology to, or having a functional fragment of the native, active length type-A neurotoxin polypeptide.

### Type B

A BTTB refers to all forms of BT which comprise the Clostridium botulinum type B neurotoxin.

BTTB may include the commercial products Myoblock (Solstice Neurosciences, USA, Canada) and Neurobloc (Solstice Neurosciences, Europe). These above mentioned products are within the scope of a BTTB according to the present invention.

BTTB may include a complex-free type-B neurotoxin. BTTB may also include a polypeptide comprising the sequence of the active type-B neurotoxin polypeptide. It may also include polypeptide with a similar identity, homology to, or comprising a functional fragment of the native, active type-B neurotoxin polypeptide.

Nucleic acid encoding BTTB may comprise a nucleic acid sequence capable of encoding the active type-B neurotoxin. It may alternatively comprise a nucleic acid sequence capable of encoding a polypeptide with a similar identity, homology to, or having a functional fragment of the native, active type-B neurotoxin polypeptide.

### Type C1

BTTC1 refers to all forms of BT which comprise the Clostridium botulinum type C1 neurotoxin. BTTC1 may include commercial products. These products are within the scope of a BTTC1 according to the present invention.

BTTC1 may include a complex-free type-C1 neurotoxin. BTTC1 may also include a polypeptide comprising the sequence of active type-C1 neurotoxin. It may also include polypeptide with a similar identity, homology to, or comprising a functional fragment of the native, active type-C1 neurotoxin polypeptide.

Nucleic acid encoding BTTC1 may comprise a nucleic acid sequence capable of encoding active type-C1 neurotoxin polypeptide. It may alternatively comprise a nucleic acid sequence capable of encoding a polypeptide with a similar identity, homology to, or having a functional fragment of the native, active type-C1 neurotoxin polypeptide.

### Type C2

BTTC2 refers to all forms of BT which comprise the Clostridium botulinum type C2 neurotoxin. BTTC2 may include commercial products. These products are within the scope of a BTTC2 according to the present invention. BTTC2 may include a complex-free type-C2 neurotoxin. BTTC2 may include component I of Clostridium botulinum type C2 neurotoxin. BTTC2 may include component II of Clostridium botulinum type C2 neurotoxin.

BTTC2 may also include a polypeptide comprising a sequence of active type-C2 neurotoxin component I. It may also include polypeptide with a similar identity, homology to, or comprising a functional fragment of the native, active type-C2 neurotoxin component I. BTTC2 may also include a polypeptide comprising a sequence of active type-C2 neurotoxin component II. It may also include polypeptide with a similar identity, homology to, or comprising a functional fragment of the native active type-C2 neurotoxin component I or component II polypeptide.

Nucleic acid encoding BTTC2 may comprise a nucleic acid sequence capable of encoding active type-C2 neurotoxin component I polypeptide. It may alternatively comprise a nucleic acid sequence capable of encoding a polypeptide with a similar identity, homology to, or having a functional fragment of the native, active type-C2 neurotoxin component I polypeptide.

Nucleic acid encoding BTTC2 may comprise a nucleic acid sequence capable of encoding active type-C2 neurotoxin component II polypeptide. It may alternatively comprise a nucleic acid sequence capable of encoding a polypeptide with a similar identity, homology to, or having a functional fragment of the native, active type-C2 neurotoxin component II polypeptide.

### Type C3

BTTC3 refers to all forms of BT which comprise the Clostridium botulinum type C3 neurotoxin. BTTC3 may include commercial products. These products are within the scope of a BTTC3 according to the present invention.

BTTC3 may include a complex-free type-C3 neurotoxin. BTTC3 may also include a polypeptide comprising the sequence of active type-C3 neurotoxin. It may also include polypeptide with a similar identity, homology to, or comprising a functional fragment of the native, active type-C3 neurotoxin polypeptide.

Nucleic acid encoding BTTC3 may comprise a nucleic acid sequence capable of encoding active type-C3 neurotoxin polypeptide. It may alternatively comprise a nucleic acid sequence capable of encoding a polypeptide with a similar identity, homology to, or having a functional fragment of the native, active type-C3 neurotoxin polypeptide.

### Type D

BTTD refers to all forms of BT which comprise the Clostridium botulinum type D neurotoxin. BTTD may include commercial products. These products are within the scope of a BTTD according to the present invention. BTTD may include a complex-free type-D neurotoxin.

BTTD may also include a polypeptide comprising a sequence of active type-D neurotoxin polypeptide. It may also include polypeptide with a similar identity, homology to, or comprising a functional fragment of the native, active type-D neurotoxin. It may also include polypeptide with a similar identity, homology to, or comprising a functional fragment of the native, active type-D neurotoxin polypeptide.

Nucleic acid encoding BTTD may comprise a nucleic acid sequence capable of encoding active type-D neurotoxin polypeptide. It may alternatively comprise a nucleic acid sequence capable of encoding a polypeptide with a similar identity, homology to, or having a functional fragment of the native, active type-D neurotoxin polypeptide.

### Type E

BTTE refers to all forms of BT which comprise the Clostridium botulinum type E neurotoxin. BTTE may include commercial products. These products are within the scope of a BTTE according to the present invention. BTTE may include a complex-free type-E neurotoxin.

BTTE may also include a polypeptide comprising a sequence of active type-E neurotoxin. It may also include polypeptide with a similar identity, homology to, or comprising a functional fragment of the native, active type-E neurotoxin polypeptide. It may also include polypeptide with a similar identity, homology to, or comprising a functional fragment of the native active type-E neurotoxin polypeptide.

Nucleic acid encoding BTTE may comprise a nucleic acid sequence capable of encoding active type-E neurotoxin polypeptide. It may alternatively comprise a nucleic acid sequence capable of encoding a polypeptide with a similar identity, homology to, or having a functional fragment of the native, active type-E neurotoxin polypeptide.

### Type F

BTTF refers to all forms of BT which comprise the Clostridium botulinum type F neurotoxin. BTTF may include commercial products. These products are within the scope of a BTTF according to the present invention. BTTF may include a complex-free type-E neurotoxin.

BTTF may also include a polypeptide comprising a sequence of active type-F neurotoxin polypeptide. It may also include polypeptide with a similar identity, homology to, or comprising a functional fragment of the native, active type-F neurotoxin polypeptide. It may also include polypeptide with a similar identity, homology to, or comprising a functional fragment of the native, active type-F neurotoxin polypeptide.

Nucleic acid encoding BTTF may comprise a nucleic acid sequence capable of encoding active type-F neurotoxin polypeptide. It may alternatively comprise a nucleic acid sequence capable of encoding a polypeptide with a similar identity, homology to, or having a functional fragment of the native, active type-F neurotoxin polypeptide.

### Type G

BTTG refers to all forms of BT which comprise the Clostridium botulinum type G neurotoxin. BTTG may include commercial products. These products are within the scope of a BTTG according to the present invention. BTTG may include a complex-free type-G neurotoxin.

BTTG may also include a polypeptide comprising a sequence of active type-G neurotoxin polypeptide. It may also include polypeptide with a similar identity, homology to, or comprising a functional fragment of the native, active type-G neurotoxin polypeptide. It may also include polypeptide with a similar identity, homology to, or comprising a functional fragment of the native, active type-G neurotoxin polypeptide.

Nucleic acid encoding BTTG may comprise a nucleic acid sequence capable of encoding active type-G neurotoxin polypeptide. It may alternatively comprise a nucleic acid sequence capable of encoding a polypeptide with a similar identity, homology to, or having a functional fragment of the native, active type-G neurotoxin polypeptide.

According to one embodiment of the invention, a BT polypeptide refers to a polypeptide having at least 80% amino acid identity, preferably 85%, 90%, 95%, or higher, up to and including 100% identity, with active BT, and which exhibits a neurotoxic activity e.g. it blocks neurotransmitter release at peripheral cholinergic nerve terminals such as the neuromuscular junction. A BT polypeptide may also be a functional fragment of active BT and which exhibits a neurotoxic activity e.g. a portion of BT which blocks neurotransmitter release at peripheral cholinergic nerve terminals such as the neuromuscular junction. A functional fragment of a BT polypeptide may comprise at least 10, 20, 30, 40, 50, 60, 70, 80, 90, or 95% of the amino acids of the sequence represented by the native sequence.

A BT polypeptide also refers to an homologous sequence of an active BT polypeptide. Where homology indicates sequence identity, means a sequence which presents a high sequence identity (e.g. more than 50%, 55%, 60%, 65%, 70%, 75%, 80%, 85%, 90%, 95% or 98% sequence identity) with the complete nucleotide or amino acid sequence of native, active BT. A functional homologue is characterized by the ability to block neurotransmitter release at peripheral cholinergic nerve terminals such as the neuromuscular junction.

Homologous sequences may comprise additions, deletions or substitutions of one or more amino acids or nucleotides, which do not substantially alter the functional characteristics of BT. That is, homologues may have at least 90% of the activity of native, active BT.

Homologous sequences of BT can also be nucleotide sequences of more than 50, 100, 200, 300, 400, 600, 800 or 1000 nucleotides which are able to hybridise to the active BT sequence under stringent hybridisation conditions (such as the ones described by SAMBROOK et al., Molecular Cloning, Laboratory Manuel, Cold Spring, Harbor Laboratory press, New York).

In an embodiment, this invention provides a pharmaceutical composition comprising at least one BT as disclosed herein as an active ingredient. Generally, the pharmaceutical compositions additionally comprise a pharmaceutically acceptable carrier diluent, excipient or carrier (collectively referred to herein as carrier materials).

In the pharmaceutical composition of the present invention, the active ingredient is typically administered in admixture with suitable carrier materials selected with respect to the intended form of administration (i.e. capsules, powders, elixirs, syrups, solutions, suspensions, emulsions, slow-release inserts, slow-release gels, implants, solutions for injection and the like).

Liquid form preparations include solutions, suspensions and emulsions. For example, D-mannitol, distilled water, p-hydroxybenzoate and the like may be included for parenteral injection solutions.

When desired or needed, suitable binders, lubricants, disintegrants, colouring agents, preservatives, buffers, anti-oxidants, coating agents, slow-release agents and the like may also be included in the pharmaceutical composition.

Preferably, the pharmaceutical composition is administered by any means for delivering BT to a tumour, or using any method to apply the composition to the region of cancerous cells or tumour.

Preferably, the pharmaceutical composition of the present invention is in a unit dosage form. In such form, the pharmaceutical composition may be in a single unit dosage form or be subdivided into suitably sized unit doses containing appropriate quantities of the active ingredient, i.e. an effective amount to achieve the desired purpose of causing the arrest or regression of cancerous cells or tumours in a host, in combination with cytotoxic therapy.

The amount of BT in a unit dose of preparation, whether administered alone or in combination with cytotoxic therapy, may be widely variable, depending upon a subject's age, weight, sex, and severity of the conditions being treated.

The specific dosage appropriate for administration is readily determined by one of ordinary skill in the art according to the factors discussed above. The dosage can also depend upon the size of the tumor to be treated, and the commercial preparation of the toxin. In regard of commercial preparations, the skilled person will be aware of the difference in activities owing to the different processes by which they are made. For example, one unit of Botox® from Allergan is said to be equivalent to three to five units of Dysport® from Ipsen. Additionally, the estimates for appropriate dosages in humans can be extrapolated from determinations of the amounts of BT required for effective treatment in non-humans. Thus, the amount of BT to be injected is proportional to the mass and level of activity of the tissue or cells to be treated. Generally, between about 0.01 and 2000 units per kg of patient weight of a BT, such as botulinum toxin type A, can be administered to effectively accomplish a toxin induced effect upon administration of the neurotoxin at or to the vicinity of the cancerous tissue. Less than about 0.01 U/kg of a botulinum toxin does not have a significant therapeutic effect while more than about 2000 U/kg or 35 U/kg of a botulinum toxin B or A, respectively, approaches a toxic dose of the specified botulinum toxin. Careful placement of the injection needle and a low volume of neurotoxin used prevents significant amounts of botulinum toxin from appearing systemically. A more preferred dose range is from about 0.01 U/kg to about 25 U/kg of a botulinum toxin, such as that formulated as BOTOX®. The actual amount of U/kg of a botulinum toxin to be administered depends upon factors such as the extent (mass) and level of activity of the i.e. cancerous cells or tissue to be treated and the administration route chosen.

According to one aspect of the invention, the dose of BT is 10⁻³ to 35 U/kg, 10⁻² to 25 U/kg, 10⁻² to 15 U/kg, 1 to 10 U/kg, 10⁻³ to 2000 U/Kg, 1 to 40000 U/Kg, 10⁻² to 200 U/kg, 10⁻¹ to 35 U/kg, 10⁻³ to 2000 U/Kg, 0.5 to 500 U/Kg, 0.5 to 1000 U/Kg, 0.5 to 2000 U/Kg, 0.5 to 3000 U/Kg, 10 to 500 U/Kg, 10 to 1000 U/Kg, 10 to 2000 U/Kg, or 10 to 3000 U/Kg. Preferably, a suitable dose will be in the range of from about 0.5 to about 500 U/kg of body weight per day.

The method of the present invention is suitable for the treatment of cancerous cells or tumours present in any subject which is a mammal such as, for example, mice, rats, monkeys, camels, goats, rabbits, livestock (e.g. cow, sheep, hen, chicken), domestic animals (e.g. cat, dog) and preferably humans.

A broad range of cancers may be treated in accordance with the present invention. These cancers include both primary and metastatic cancers. Specific types of cancers that can be treated include, but are not limited to, gastric cancer, lung cancer, ovarian cancer, liver cancer, uterine cancer, thyroid cancer, pancreatic cancer, lingual cancer, head and neck, bile duct cancer, and other various types of cancer. Treatable cancers also include prostate cancer, rectal cancer, mammary cancer, skin cancer, colon cancer, and CNS cancer

According to the present invention, a cytotoxic therapy is any treatment which leads to cell death. Cytotoxic therapy is well known in the art for treating cancer. Cytotoxic therapy can be localised treatment e.g. radiotherapy, laser treatment, magic bullet (e.g. antibody-toxin constructs) or systemic e.g. chemotherapy using cellular toxins. Generally chemotherapy agents are more quickly absorbed by rapidly dividing cells. They, therefore, discriminate against healthy cells by their rate of uptake.

According to one embodiment of the present invention, a cytotoxic therapy is radiotherapy. In this aspect, the invention provides a treatment for cancers comprising administering to a subject a pharmaceutical comprising BT in an effective amount in combination with radiotherapy.

The inventors have found that administering BT to cancerous cells or a tumour in combination with radiotherapy is synergistically effective in treating said cells in comparison with radiotherapy only, or with BTTA only. The studies by the present inventors found BTTA alone had no effect on the tumours tested. The inventors investigated two the tumor models—one fibrosarcoma and the other hepatocarcinoma. When BTTA was injected into the tumour without co-treatment, there was no modification of the tumor growth nor induction of apoptosis. In addition, when tumour cells were incubated in the presence of BTTA, there was no cell death. Furthermore, no clonogenic death was observed when tumor cells are incubated in the presence of BTTA. Therefore, it is clear that even in the absence of direct effect of BBTA on tumor cells, the cytotoxic treatment such as radiotherapy or chemotherapy is more effective than BTTA alone; the combination of BT and cytotoxic treatment is surprisingly efficacious.

Radiotherapy may be administered according to the present invention in a variety of fashions. For example, radiation may be electromagnetic or particulate in nature. Electromagnetic radiation useful in the practice of this invention includes, but is not limited to, x-rays and gamma rays. Particulate radiation useful in the practice of this invention includes, but is not limited to, electron beams, proton beams, neutron beams, alpha particles, and negative pi mesons. Radiation may be delivered using conventional radiological treatment apparatus and methods. Additional information regarding radiation treatments suitable for use in the practice of the present invention may be found in Textbook of Radiation Oncology (see Steven A. Leibel et al., published by W. B. Saunders Company, 1998). Radiation may also be delivered by other methods such as targeted delivery, for example by radioactive “seeds”, or by systemic delivery of targeted radioactive conjugates. Other conventional radiation delivery methods also may be used in the practice of this invention.

The amount of radiation may be variable. In a preferable embodiment, radiation may be administered in amount effective to cause the arrest or regression of cancerous cells or tumours in a subject, when the radiation is co-administered with a pharmaceutical composition comprising at least one BT. The radiation may be administered in a variety of treatment plans including the amount and duration of radiation. Choice of the radiation treatment plan may be made by one of skill in the art, depending upon the appropriate course of therapy.

According to one embodiment of the invention, a pharmaceutical composition is administered prior to radiotherapy. The time interval between administration of the composition and radiotherapy can be determined by the skilled person in view of the effects of the treatment. For example, radiotherapy may commence up to 336, 312, 288, 264, 240, 216, 192, 168, 144, 120, 96, 72, 48, 24, 20, 16, 12, 8, 4, 2, 1, 0.5, 0.4, 0.3, 0.2, or 0.1 hours after administering the pharmaceutical composition.

According to one embodiment of the invention, a pharmaceutical composition is administered during radiotherapy. Typically the administration will start and finish within the radiotherapy treatment session period.

According to one embodiment of the present invention, a cytotoxic therapy is chemotherapy. According to another embodiment of the present invention, a cytotoxic therapy is systemic chemotherapy.

Another aspect of the invention is a pharmaceutical composition comprising one or more BTs and at least one chemotherapy agent.

Another aspect of the invention is a pharmaceutical composition comprising one or more BTs and at least one chemotherapy agent for the treatment of cancerous cells or tumours in a subject.

According to one aspect of the invention, a chemotherapy agent is administered simultaneously, separately or sequentially in respect of a pharmaceutical composition comprising one or more BTs.

Another aspect of the invention is a pharmaceutical composition comprising one or more BTs and at least one chemotherapy agent for simultaneous, separate or sequential administration to a subject.

One aspect of the invention is a method for treating cancer comprising administering to an individual an effective amount of BT and at least one chemotherapy agent, simultaneously, separately or sequentially.

By simultaneous administration means BT and chemotherapy agent are administered to a subject at the same time. For example, as a mixture or a composition comprising said components. An example is as a solution comprising the components of interest.

By separate administration means BT and chemotherapy agent are administered to a subject at the same time or substantially the same time. The components may be present in a kit as separate, unmixed preparations. For example, BT and chemotherapy agent may be present in the kit as individual vials. The inhibitors may be administered to the subject by separate injections at the same time, or injection directly following the other.

By sequential administration means BT and chemotherapy agent are administered to a subject sequentially. BT and chemotherapy agent may be present in a kit as separate, unmixed preparations. There is a time interval between doses. For example, one component might be administered up to 336, 312, 288, 264, 240, 216, 192, 168, 144, 120, 96, 72, 48, 24, 20, 16, 12, 8, 4, 2, 1, 0.5, 0.4, 0.3, 0.2, or 0.1 hours after the other component.

In sequential administration, one component may be administered once, or any number of times and in various doses before and/or after administration of another component. Sequential administration may be combined with simultaneous or sequential administration.

A chemotherapy agent according to the present invention is an agent effective in causing the arrest or regression of cancerous cells or tumours in a subject. Preferably a chemotherapy agent is one which is cytotoxic and which affects rapidly dividing cells. Preferably it is systemically administered. Chemotherapy agents include, for example, taxol, gemcitabine and cis-platin. BT is not considered a chemotherapy agent according to the present invention.

The inventors have found that administering BT to cancerous cells or tumour in combination with chemotherapy is synergistically effective in treating said cells in comparison with radiotherapy only. Administering BT sensitises cancerous cells or tumour to subsequent treatment with chemotherapy.

A pharmaceutical composition of the present invention is preferably administered to the cancerous cells or tumour. Preferably delivery is localised in the vicinity of the cancer. Where the cancer is a tumour, administration may be by injection into the tumour, application to the surface of the tumour, injection in a blood vessel supplying the tumour or any known method of locally administering the pharmaceutical composition.

A pharmaceutical composition of the present invention may comprise nucleic acid capable of encoding BT, as already mentioned above. Said nucleic acid may replace the BT in the above mentioned embodiments, or may be provided in addition. Thus, one embodiment of the present invention is a composition comprising a nucleic acid capable of encoding a BT for the treatment of cancerous cells or tumours. A nucleic may be administered in the form of a vector, as an expressing bacterial strain or in any carrier known the skilled person suitable for the expression nucleic acid.

One embodiment of the present invention is a pharmaceutical composition comprising at least one Botulinum toxin, BT, for the preparation of a medicament for sensitising a cancer to treatment with cytotoxic therapy.

Another embodiment of the present invention is a use as described above, wherein said composition is administered locally to cancerous cells or tumours.

Another embodiment of the present invention is a use as described above, wherein the cytotoxic therapy is radiotherapy.

Another embodiment of the present invention is a use as described above, wherein the pharmaceutical composition is administered prior to radiotherapy.

Another embodiment of the present invention is a use as described above, wherein the pharmaceutical composition is administered during radiotherapy.

Another embodiment of the present invention is a use as described above, wherein a cytotoxic therapy is chemotherapy.

Another embodiment of the present invention is a use as described above, wherein a cytotoxic therapy is systemic chemotherapy.

Another embodiment of the present invention is a use as described above, wherein a chemotherapy agent is administered simultaneously, separately or sequentially in respect of said pharmaceutical composition.

Another embodiment of the present invention is a use as described above, wherein said chemotherapy comprises administering a chemotherapy agent effective in causing the arrest or regression of cancerous cells or tumours in a subject.

Another embodiment of the present invention is a use as described above, wherein said chemotherapy agent is any of taxol, gemcitabine or cis-platin or a combination thereof.

Another embodiment of the present invention is a use as described above, wherein the BTs of a composition comprising two or more BTs are administered simultaneously, separately or sequentially.

Another embodiment of the present invention is a use as described above, wherein a pharmaceutical composition further comprises a suitable carrier material.

Another embodiment of the present invention is a use as described above, wherein the cancer is any of gastric cancer, lung cancer, ovarian cancer, prostate cancer, liver cancer, uterine cancer, thyroid cancer, pancreatic cancer, lingual cancer, bile duct cancer, rectal cancer, mammary cancer, skin cancer, colon cancer, head and neck cancer or CNS cancer.

Another embodiment of the present invention is a pharmaceutical composition comprising at least one BT and at least one chemotherapy agent.

Another embodiment of the present invention is a pharmaceutical composition as described above, for simultaneous, separate or sequential administration to a subject.

Another embodiment of the present invention is a use or pharmaceutical composition as described above wherein a polypeptide of a BT is replaced with a nucleic acid capable of encoding said polypeptide.

Another embodiment of the present invention is a use or pharmaceutical composition as described above wherein said nucleic acid is capable of encoding a homologue or functional fragment of said BT.

Another embodiment of the present invention is a use or pharmaceutical composition as described above wherein the BT comprises Clostridium botulinum type A neurotoxin, BTTA.

Another embodiment of the present invention is a use or pharmaceutical composition as described above, wherein the BTTA comprises an active Clostridium botulinum type A neurotoxin polypeptide.

Another embodiment of the present invention is a use or pharmaceutical composition as described above, wherein said BTTA comprises a homologue or functional fragment of an active Clostridium botulinum type A neurotoxin polypeptide.

Another embodiment of the present invention is a use or pharmaceutical composition as described above, wherein the BTTA is Nc-224 or Nc-270.

Another embodiment of the present invention is a use or pharmaceutical composition as described above, wherein the BTTA is any of Botox®, Botox® Cosmetic, Vistabel®, Dysport®, Reloxin™, Clostridium botulinum type A toxins prepared by Mentor Corporation, Xeomin®, Linurase®, CBTX-A® or Neuronox® or any pharmaceutical product comprising BTTA.

Another embodiment of the present invention is a use or pharmaceutical composition as described above, wherein said nucleic acid comprises a nucleic acid sequence capable of encoding an active Clostridium botulinum type B neurotoxin polypeptide.

Another embodiment of the present invention is a use or pharmaceutical composition as described above, wherein the BT comprises Clostridium botulinum type B neurotoxin, BTTB.

Another embodiment of the present invention is a use or pharmaceutical composition as described above, wherein the BTTB comprises an active Clostridium botulinum type B neurotoxin polypeptide.

Another embodiment of the present invention is a use or pharmaceutical composition as described above, wherein said BTTB comprises a homologue or functional fragment of an active Clostridium botulinum type B neurotoxin polypeptide.

Another embodiment of the present invention is a use or pharmaceutical composition as described above, wherein the BTTB is Myoblock or Neurobloc.

Another embodiment of the present invention is a use or pharmaceutical composition as described above, wherein said nucleic acid comprises a nucleic acid sequence capable of encoding an active Clostridium botulinum type B neurotoxin polypeptide.

Another embodiment of the present invention is a use or pharmaceutical composition as described above, wherein the BT comprises Clostridium botulinum type C1 neurotoxin, BTTC1.

Another embodiment of the present invention is a use or pharmaceutical composition as described above, wherein the BTTC1 comprises an active Clostridium botulinum type C1 neurotoxin polypeptide.

Another embodiment of the present invention is a use or pharmaceutical composition as described above, wherein said BTTC1 comprises a homologue or functional fragment of an active Clostridium botulinum type C1 neurotoxin polypeptide.

Another embodiment of the present invention is a use or pharmaceutical composition as described above, wherein said nucleic acid comprises a nucleic acid sequence capable of encoding an active Clostridium botulinum type C1 neurotoxin polypeptide.

Another embodiment of the present invention is a use or pharmaceutical composition as described above, wherein the BT comprises Clostridium botulinum type C2 neurotoxin, BTTC2.

Another embodiment of the present invention is a use or pharmaceutical composition as described above, wherein said BTTC2, comprises BTTC2 component II.

Another embodiment of the present invention is a use or pharmaceutical composition as described above, wherein said BTTC2, comprises BTTC2 component II.

Another embodiment of the present invention is a use or pharmaceutical composition as described above, wherein the BTTC2 component I comprises an active Clostridium botulinum type C2 neurotoxin component I polypeptide.

Another embodiment of the present invention is a use or pharmaceutical composition as described above, wherein said BTTC2 component I comprises a homologue or functional fragment of an active Clostridium botulinum type C2 neurotoxin component I polypeptide.

Another embodiment of the present invention is a use or pharmaceutical composition as described above, wherein the BTTC2 c component II comprises an active Clostridium botulinum type C2 neurotoxin component II polypeptide.

Another embodiment of the present invention is a use or pharmaceutical composition as described above, wherein said BTTC2 component II comprises a homologue or functional fragment of an active Clostridium botulinum type C2 neurotoxin component II polypeptide.

Another embodiment of the present invention is a use or pharmaceutical composition as described above, wherein said nucleic acid comprises a nucleic acid sequence capable of encoding an active BTTC2 component I or II polypeptide.

Another embodiment of the present invention is a use or pharmaceutical composition as described above, wherein the BT comprises Clostridium botulinum type C3 neurotoxin, BTTC3.

Another embodiment of the present invention is a use or pharmaceutical composition as described above, wherein the BTTC3 comprises an active Clostridium botulinum type C3 neurotoxin polypeptide.

Another embodiment of the present invention is a use or pharmaceutical composition as described above, wherein said BTTC3 comprises a homologue or functional fragment of an active Clostridium botulinum type C3 neurotoxin polypeptide.

Another embodiment of the present invention is a use or pharmaceutical composition as described above, wherein said nucleic acid comprises a nucleic acid sequence capable of encoding an active BTTC3 polypeptide.

Another embodiment of the present invention is a use or pharmaceutical composition as described above, wherein the BT comprises Clostridium botulinum type D neurotoxin, BTTD.

Another embodiment of the present invention is a use or pharmaceutical composition as described above, wherein the BTTD comprises an active Clostridium botulinum type D neurotoxin polypeptide.

Another embodiment of the present invention is a use or pharmaceutical composition as described above, wherein said BTTD comprises a homologue or functional fragment of an active Clostridium botulinum type D neurotoxin polypeptide.

Another embodiment of the present invention is a use or pharmaceutical composition as described above, wherein said nucleic acid comprises a nucleic acid sequence capable of encoding an active BTTD polypeptide.

Another embodiment of the present invention is a use or pharmaceutical composition as described above, wherein the BT comprises Clostridium botulinum type E neurotoxin, BTTE.

Another embodiment of the present invention is a use or pharmaceutical composition as described above, wherein the BTTE comprises an active Clostridium botulinum type E neurotoxin polypeptide.

Another embodiment of the present invention is a use or pharmaceutical composition as described above, wherein said BTTE comprises a homologue or functional fragment of an active Clostridium botulinum type E neurotoxin polypeptide.

Another embodiment of the present invention is a use or pharmaceutical composition as described above, wherein said nucleic acid comprises a nucleic acid sequence capable of encoding an active BTTE polypeptide.

Another embodiment of the present invention is a use or pharmaceutical composition as described above, wherein the BT comprises Clostridium botulinum type F neurotoxin, BTTF.

Another embodiment of the present invention is a use or pharmaceutical composition as described above, wherein the BTTF comprises an active Clostridium botulinum type F neurotoxin polypeptide.

Another embodiment of the present invention is a use or pharmaceutical composition as described above, wherein said BTTF comprises a homologue or functional fragment of an active Clostridium botulinum type F neurotoxin polypeptide.

Another embodiment of the present invention is a use or pharmaceutical composition as described above, wherein said nucleic acid comprises a nucleic acid sequence capable of encoding an active BTTF polypeptide.

Another embodiment of the present invention is a use or pharmaceutical composition as described above, wherein the BT comprises Clostridium botulinum type G neurotoxin, BTTG.

Another embodiment of the present invention is a use or pharmaceutical composition as described above, wherein the BTTG comprises an active Clostridium botulinum type G neurotoxin polypeptide.

Another embodiment of the present invention is a use or pharmaceutical composition as described above, wherein said BTTG comprises a homologue or functional fragment of an active Clostridium botulinum type G neurotoxin polypeptide.

Another embodiment of the present invention is a use or pharmaceutical composition as described above, wherein said nucleic acid comprises a nucleic acid sequence capable of encoding an active BTTG polypeptide.

Another embodiment of the present invention is a method for enhancing radiotherapy treatment of cancerous cells or tumours comprising the step of administering to said cells or tumours one or more BTs.

Another embodiment of the present invention is a method for enhancing chemotherapy treatment of cancerous cells or tumours comprising the step of administering to said cells or tumours one or more BTs.

Another embodiment of the present invention is a method of sensitising cancerous cells or tumours to systemic radiotherapy, comprising administering to said cells or tumours one or more BTs.

Another embodiment of the present invention is a method of sensitising cancerous cells or tumours to systemic chemotherapy, comprising administering to said cells or tumours one or more BTs.

Another embodiment of the present invention is a method of increasing the uptake of an active compound in cancerous cells or tumours, comprising administering to said cells or tumours one or more BTs.

Another embodiment of the present invention is a method as described above, wherein said composition is administered locally to cancerous cells or tumours.

Another embodiment of the present invention is a method as described above, wherein the cytotoxic therapy is radiotherapy.

Another embodiment of the present invention is a method as described above, wherein the pharmaceutical composition is administered prior to radiotherapy.

Another embodiment of the present invention is a method as described above, wherein the pharmaceutical composition is administered during radiotherapy.

Another embodiment of the present invention is a method as described above, wherein a cytotoxic therapy is chemotherapy.

Another embodiment of the present invention is a method as described above, wherein a cytotoxic therapy is systemic chemotherapy.

Another embodiment of the present invention is a method as described above, wherein a chemotherapy agent is administered simultaneously, separately or sequentially in respect of said pharmaceutical composition.

Another embodiment of the present invention is a method as described above, wherein said chemotherapy comprises administering a chemotherapy agent effective in causing the arrest or regression of cancerous cells or tumours in a subject.

Another embodiment of the present invention is a method as described above, wherein said chemotherapy agent is gemcitabine, taxol or cis-platin or a combination thereof.

Another embodiment of the present invention is a method as described above, wherein the BTs of a composition comprising two or more BT are administered simultaneously, separately or sequentially.

Another embodiment of the present invention is a method as described above, wherein a pharmaceutical composition further comprises a suitable carrier material.

Another embodiment of the present invention is a method as described above, wherein the cancer is any of gastric cancer, lung cancer, ovarian cancer, prostate cancer, liver cancer, uterine cancer, thyroid cancer, pancreatic cancer, lingual cancer, bile duct cancer, rectal cancer, mammary cancer, skin cancer, colon cancer, head and neck cancer or CNS cancer.

Another embodiment of the present invention is a method as described above, wherein a polypeptide of BT is replaced with a nucleic acid capable of encoding said polypeptide.

Another embodiment of the present invention is a method as described above, wherein said nucleic acid is capable of encoding a homologue or functional fragment of said BT.

Another embodiment of the present invention is a method as described above, wherein a BT is as defined above.

### EXAMPLES

The present invention is illustrated by the following non-limiting examples.

### Example 1

### Experiment 1

### In Vivo Tumour Preparation

Two different tumour models were implanted in the thigh of mice: Syngeneic FSa II fibrosarcoma tumour cells were injected intramuscularly in male C3H/HeOuJlco mice and a Transplantable mouse Liver Tumour (TLT) model injected into NMRI mice. Both tumor models were previously characterised by the inventors for assessing the effect of treatments which potentiate radiotherapy and chemotherapy. Tumors were measured daily with an electronic caliper. For all experiments, tumor-bearing mice were anesthetized using isoflurane (2.5% for induction, 1% for maintenance). All animal experiments were conducted in accordance with national animal care regulations.

BTTA (i.e. Botox®) or saline solution (control) was injected when tumours reached a diameter of 6.5±1.0 mm. Botox® (Allergan, Antwerp, Belgium) was dissolved in saline water and was given via intratumoural injections of 40 μl total. A 20 μl injection was performed at two different places in the tumours (2 injections of 20 μl, corresponding to a total dose of 29 Ukg⁻¹). For all experiments, tumour-bearing mice were anesthetized using isoflurane (2.5% for induction, 1% for maintenance).

### Experiment 2

### Tumour Oxygenation

Electronic Paramagnetic Resonance (EPR) Oximetry using charcoal (CX0670-1, EM Science, Gibbstown, N.J.) as oxygen sensitive probe was used to evaluate tumour oxygenation changes after treatment with BTTA. EPR spectra were recorded using an EPR spectrometer (Magnettech, Berlin, Germany) with a low frequency microwave bridge operating at 1.2 GHz and extended loop resonator. Mice were injected in the centre of the tumour 1 day before measurement using the suspension of charcoal (suspension in saline containing 3% Arabic gum, 100 mg/ml, 50 μl injected, 1 to 25 μm particle size). The localized EPR measurements correspond to an average of pO₂ values in a volume of ˜10 mm³. In order to avoid any acute effect of the treatment, data acquisition was made before the injection of BTTA or saline and then on a daily basis.

### Experiment 3

### Flow Measurements

### Patent Blue Staining.

Patent Blue (Sigma-Aldrich, Bornem, Belgium) was used to obtain a rough estimate of the tumour perfusion three days after administration of the BTTA or vehicle. This technique involves the injection of 200 μl of Patent Blue (1.25%) solution into the tail vein of the mice. After 1 min, a uniform distribution of the staining through the body was obtained and mice were sacrificed. Tumours were carefully excised and cut in two size-matched halves. Pictures of each tumour cross-section were taken with a digital camera. In order to compare the stained with unstained area, an in house program running on IDL (Interactive Data Language, RSI, Boulder, Colo.) was developed. For each tumour, a region of interest (stained area) was defined on the two pictures and the percentage of stained area of the whole cross-section was determined. The mean of the percentage of the two pictures was then calculated and was used as an indicator of tumour perfusion.

### Dynamic Contrast Enhanced Magnetic Resonance Imaging (DCE MRI).

Using DCE MRI, it is possible to provide parametric maps that reflect the plasma volume fraction, the permeability and the rate of efflux. To generate these parametric maps, MRI was performed with a 4.7 Tesla (200 MHz, 1H), 40 cm inner diameter bore system (Bruker Biospec, Ettlingen, Germany). T2-weighted anatomical images were acquired using a fast spin echo sequence (repetition time (TR)=4 s, effective echo time (TE)=50.5 ms). A single, 1.6 mm thick slice passing through the tumour centre was localized. A birdcage radio-frequency coil with an inner diameter of 70 mm was used for radio-frequency transmission and reception. For the dynamic contrast-enhanced MRI study, two axial (transverse) slices were selected: one was centred on the kidneys and the other was positioned on the tumour. T1-weighted gradient-recalled echo images were obtained with the following parameters: TR=40 ms, TE=4.9 ms, 1.6 mm slice thickness, flip angle=90°, matrix=64×64, FOV=6 cm, 25 kHz receiver bandwidth, resulting in an acquisition time of 2.56 s per scan. The contrast agent was a rapid-clearance blood pool agent, P792 (Vistarem®, Laboratoire Guerbet, Aulnay sous Bois, France). P792 (mw: 6.47 kDa) is a monogadolinium macrocyclic compound based on a Gd-DOTA structure substituted by hydrophilic (dextran) arms. Its R1 relaxivity in 37° C. HSA, 4% at 4.7 T is 9.0 mM−1s−1 (data communicated by Guerbet). P792 was injected at dose of 0.042 mmol Gd/kg as recommended by the company and published studies. The DCE study was performed using the following protocol: After 12 baseline images had been acquired, P792 was administered intravenously within 2 s (50 μl/40 g mouse) and the enhancement kinetics were continuously monitored for 8 min (200 total scans). This allowed sampling of the signal intensity curve often enough to track the fast rise in tissue enhancement for viable tumour following bolus arrival. Immediately after this, a slower DCE data set was acquired to monitor the washout of the contrast agent. For this second set, 60 scans were acquired at a temporal resolution of 60 s (1 hour total).

### Miles Assay

The Evans Blue (EB) technique was used to measure the tumor vasopermeability to macromolecules. This technique provides a good estimate of the extravasation and interstitial accumulation of albumin as EB dye makes a complex with the negatively charged intravascular albumin by electrostatic combination. Briefly, EB (2.5 mg/ml in NaCl 0.9%) was administered by IV (10 mg/kg) in control and treated mice. The dye was allowed to circulate for one hour before sacrifice of the animals. Tumors were then excised, weighed and put in formamide (2 ml/tumor) for 24 hours at 55° C. for EB extraction. The EB concentrations were determined by spectrophotometry at 620 nm and expressed in terms of concentration per gram of tumor tissue.

### Experiment 4

### Kinetic Analysis

Pixel-by-pixel values for K^{trans} (influx volume transfer constant, from plasma into the interstitial space, units of min⁻¹), Vₚ (blood plasma volume per unit volume of tissue, unitless), and Kₑₚ (fractional rate of efflux from the interstitial space back to blood, units of min⁻¹) in tumor were calculated via tracer kinetic modeling of the dynamic contrast-enhanced data, and the resulting parametric maps for K^{trans}, Vₚ, and Kₑₚ, were generated. Statistical significance for Vₚ or K^{trans} identified “perfused” pixels (i.e. pixels to which the contrast agent P792 had access).

DCE MRI raw data were zero-filled and 2D Fourier transformed, resulting in an in-plane resolution of 128×128. An operator-defined region of interest encompassing the tumour was analysed on a pixel-by-pixel basis to obtain parametric maps. Pixels showing either no signal enhancement or linear increase of SI were excluded from the analysis. This was achieved by identifying voxels with statistically significant variations in T1 weighted signal intensity using power spectrum analysis. Using cluster analysis, voxels for which typical signal enhancement curves were observed were then selected for pharmacokinetic analysis.

Contrast agent concentration as a function of time after P792 injection (C(t)) was estimated by comparing the tumour signal intensity as a function of time (S(t)) with the signal intensity in a reference tissue (muscle) with known T1. Assuming that signal intensity changes linearly as a function of contrast media concentration (T1 weighted sequence, short TE, TR<<T1), then Equation (I) applies:

$$C ( t ) = \frac{1}{{R}_{1} {T}_{1 ( muscle )}} \frac{S ( t ) - S ( 0 )}{{S}_{muscle} ( 0 )} \qquad ( l )$$

where R1 is the longitudinal relaxivity of the contrast agent (assumed to be equal to that in HSA 4%) and the T1 of muscle is assumed to be 900 ms. The tracer concentration changes were fitted to a two-compartment pharmacokinetics model. In this model, the contribution of the tracer in the blood plasma to the total tissue concentration is included (negligible in blood-brain barrier lesions but often significant in tumours) and different permeability constants for flux into and out of the extravascular extracellular space (EES) are allowed. The model assumes that the tracer is well-mixed throughout the compartments (tumour regions with high interstitial fluid pressure might not meet this condition) and that there is a fast exchange of all mobile ¹H within the tissue. The model also assumes that the increase in T₁ relaxation rate is proportional to the concentration of the tracer.

The equation describing the tissue concentration as a function of time is shown in Equation (II):

$$C ( t > t 0 ) = {K}_{in}^{Trans} A 0 \cdot \frac{{exp}^{- \frac{{K}_{out}^{Trans}}{{v}_{e}} t} - {exp}^{- k 1 t}}{k 1 - \frac{{K}_{out}^{Trans}}{{v}_{e}}} + {v}_{p} \cdot A 0 \cdot {exp}^{- k 1 t} \qquad ( ll )$$

where K^{Trans}ᵢₙ is the influx volume transfer constant (into EES from plasma), K^{Trans}ₒᵤₜ is the efflux volume transfer constant (from EES back to plasma), vₑ is the volume of extravascular extracellular space per unit volume of tissue, and vₚ is the blood plasma volume per unit volume of tissue. K^{Trans}ₒᵤₜ and vₑ can not be estimated separately, thus only kₑₚ, the ratio K^{Trans}ₒᵤₜ/vₑ, is calculated. kₑₚ is the fractional rate of efflux from the interstitial space back to the blood. The constants used in the fitting are the maximum concentration of P792 in the plasma (A0), the blood decay rate (k1), and the time to the maximum tracer plasma concentration t0. It is assumed that the rapid enhancement phase (from t=0 to t0) is primarily due to intravascular contrast media during the first pass of the contrast media bolus, while the slower phase is due to leakage into the extracellular space. A universal t0 time value was estimated for each mouse from the kidney data. Additionally, the decay rate of the contrast agent in the blood stream was estimated from the enhancement kinetics in one or two selected renal cortex voxels showing early and large [P792] signal enhancement, presumably reflecting pronounced arterial perfusion. A monoexponential function was fit to the [P792] kidney. Fitting was performed using a Levenberg-Marquardt non-linear least-squares procedure. Parametric images for K^{Trans}ₒᵤₜ, vₚ, and kₑₚ were computed, with only the statistically significant parameter estimates being displayed.

### Experiment 5

### Irradiation and Tumour Regrowth Delay Assay

The FSaII tumour-bearing leg was locally irradiated with 20 Gy of 250 kV X-rays (RT 250; Philips Medical Systems). The tumour was centered in a 3 cm diameter circular irradiation field. A single dose irradiation of 20 Gy 3 days after the single BTTA intratumoural injection was realized. After treatment, tumour growth was determined daily by measuring transversal and antero-posterior tumour diameters until they reached a double size, time at which the mice were sacrificed. A linear fit was obtained between 8 and 16 mm which allowed a determination of the time to reach a particular size for each mouse.

### Experiment 6

### Chemotherapy and Tumor Regrowth Delay Assay

BTTA-treated mice received a single dose (50 mg/kg via 100 μl IP) of cyclophosphamide, an alkylating agent. Regrowth delay experiments in TLT have shown that this dose of 50 mg/kg is just below the efficacy threshold for this product (experiments performed with doses ranging from 250 to 10 mg/kg).

### Experiment 7

### Clonogenicity Assay

FSaII tumours in mice were dissected in a sterile environment and gently pieced in McCoy's medium. The cell suspension was filtered (100 micrometer-sized pore nylon filter, Millipore, Brussels, Belgium), centrifuged (5 min, 450 g, 4 deg C.), and cells were set to culture in DMEM containing 10% fetal bovine serum. Confluent cells were treated with BTTA (0.73 U/10⁸ cells) 4 hours before being irradiated at 2 Gy. To assess the cell radiosensitivity, trypan blue exclusion dye method and a clonogenic cell survival assay were performed. For the former, the cells were counted for viability twenty-four hours after irradiation. For the latter, the cells were washed and reincubated in the conditioned medium without drug twenty-four hours after irradiation. After a 7-day incubation in a humidified 5% CO₂ atmosphere at 37 deg C., the dishes were stained with crystal violet and colonies with >50 cells were counted. The experiments were carried out in triplicate.

### Experiment 7

### Apoptotic Activity

The apoptotic activity inside tumours was assessed by measuring the activity of caspase-3, a well-known effector involved in the apoptosis induced by chemotherapeutic or radiotherapeutic treatments. The activation of caspase-3 was measured by immunoblotting, and the results were confirmed by measuring the cleavage of PARP, a polypeptide cleaved during apoptosis process. Tumours were dissected, minced, put in an extraction buffer solution (TRIS: 10 mM, EDTA: 1 mM, Sucrose: 250 mM, PMSF: 0.1 mM, NaF: 10 mM, Na₃VO₄: 1 mM, supplemented with protease inhibitor cocktail (Complete Mini, Roche applied Science—Mannheim, Germany)—pH: 7.4) and homogenized with a Potter-Elvehjem tissue grinder. The tumor homogenates were centrifuged at 10 000 g for 20 minutes and the supernatant fraction saved for analysis. From these homogenates, protein concentration was determined by the Bradford protein assay. Equal amounts of proteins (50 μg) were subjected to SDS-PAGE (6% and 15% separating gels, respectively for PARP and caspase-3 detection) followed by electroblot to nitrocellulose membranes. The membranes were blocked 1 h in TBS buffer (pH 7.4) containing 5% powdered milk protein, followed by an incubation of 2 hours with diluted antibodies in a fresh solution of powdered milk protein (1% w/v in TBS buffer). The membranes were washed and incubated for 60 min with a dilution of secondary antibody coupled with horseradish peroxidase. Anti-PARP and anti-caspase-3 rabbit polyclonal antibodies were diluted by 1:200 and goat anti-rabbit polyclonal antibody, by 1:10 000. They were purchased respectively from Santa-Cruz (CA, USA) and Chemicon Inc. (CA, USA). The quantification of the Western Blot bands was performed with by densitometry (Image Master V1.1, Pharmacia Biotech).

### Experiment 8

### Myogenic Tone Measurements

Segments of the co-opted saphenous arteries (2 mm in length), were carefully dissected. For each tumour, two adjacent segments were mounted in a multi wire-myograph (610M, DMT, Aarhus, Denmark). Briefly, two 40 μm wires were threaded through the lumen of the vessel segment. One wire was attached to a stationary support driven by a micrometer, while the other was attached to an isometric force transducer. The bath of the myograph was filled with physiological salt solution (PSS, composition in mmol/L: NaCl, 120; KCl, 5.9; NaHCO₃, 25; dextrose, 17.5; CaCl₂, 2.5; MgCl₂, 1.2; NaH₂PO₄, 1.2 (pH 7.4)), gassed and maintained at 37° C. After mounting, vessels were maintained under zero force for 45 to 60 min. A passive diameter-tension curve was constructed. The vessel was set at a tension equivalent to that generated at 90% of the diameter of the vessel under a transmural pressure of 100 mmHg. The viability of the vessels was assessed by measuring the contractile response to a depolarising solution (PSS where 100 mmol/L KCl replaced NaCl stoechiometrically). After washing, the vessels (n=4) were incubated in the presence of BTTA (0.12 U/ml) for 2 hours, while the matched-controls (adjacent segments) were kept in PSS+solvent. All vessels were then challenged with a high KCl solution (KCl 40 mmol/L) in order to depolarize smooth muscle cells of the media and nerve endings; thereby activating the Ca²⁺-dependent release of neurotransmitter. The amplitude of the neurotransmitter release was estimated by measuring the relaxation to an α-adrenoceptor blocker (phentolamine) or to a cholinergic antagonist (atropine) for noradrenaline and acetycholine, respectively.

### Statistical Analysis

Results are given as means±SEM values from n animals. Comparisons between groups were made with Student's two-tailed t-test or two ways ANOVA where appropriate, and a P value less than 0.05 was considered significant.

### Result 1

### BTTA Increases Tumor Oxygenation and Perfusion

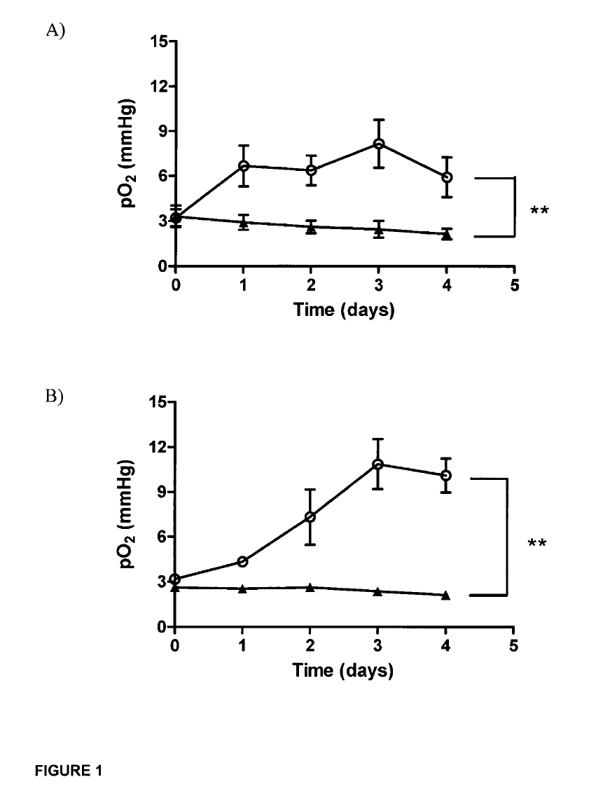

In order to determine the effect of BTTA on the tumor oxygenation as well as the kinetics of the effect, the partial pressure of oxygen (pO2) was measured daily after intra-tumoral injection of BTTA in two different sites. As shown in FIG. 1, the pO2, which was very low in the FSaII tumors before the treatment (3.2±0.5 mmHg, n=10), significantly increased after administration of BTTA, with a maximal pO2 reached after 3 days (8.2±1.6 mmHg). The tumor pO2 was found to be statistically different between BTTA treated tumors and controls (two way ANOVA). A similar effect was observed in transplantable liver tumors (TLT) (10.9±1.7 mmHg on day 3 versus 3.2±0.3 on day 0, n=4). Therefore, all further experiments for the characterization of the tumor micro-environment and the establishment of the therapeutic relevance of the administration of BTTA were conducted on day 3.

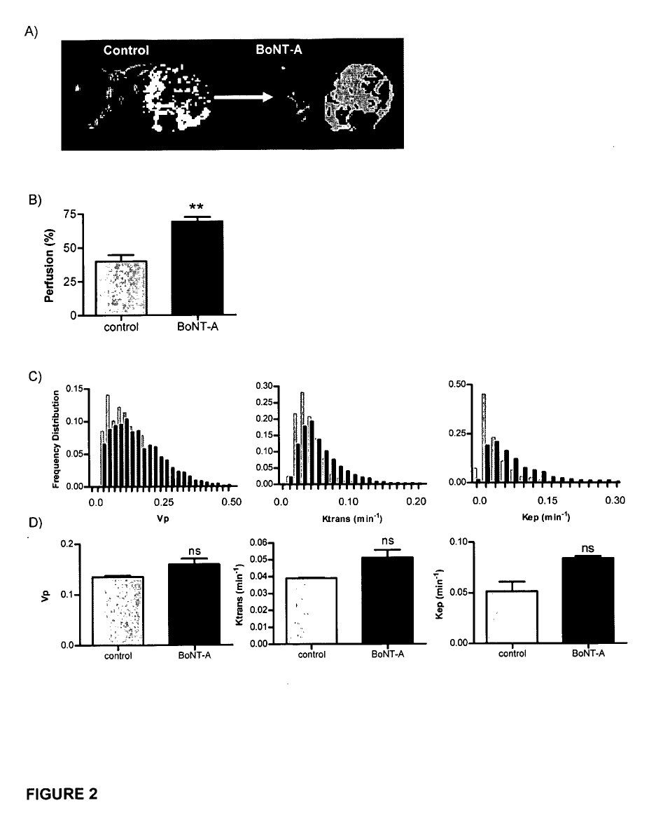

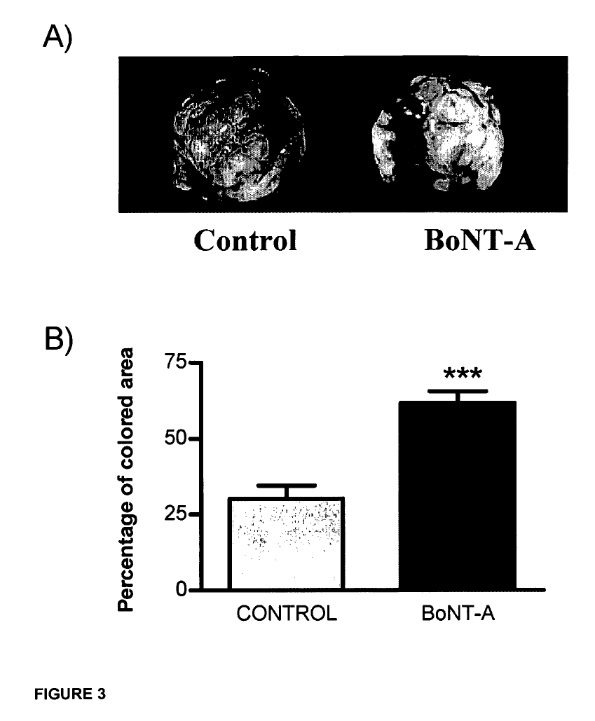

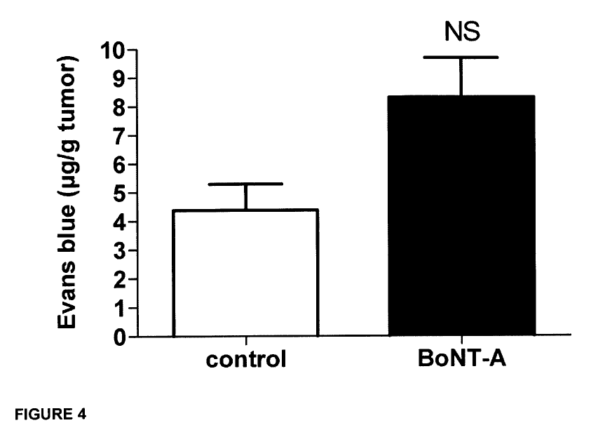

Tumor perfusion was monitored in the FSaII tumor model on day 3 after BTTA administration via dynamic contrast-enhanced MRI at 4.7 T using IV injection of the rapid-clearance blood pool agent P792 (Vistarem®). The pixel-by-pixel analysis generated “perfusion maps” (using the values for Vₚ, the blood plasma volume per unit volume of tissue), and “permeability maps” (using the values for K^{trans}, the influx volume transfer constant, from plasma into the interstitial space and Kₑₚ, the efflux volume transfer constant from the interstitial space back to plasma). Moreover, the kinetics analysis identified “perfused pixels” (i.e. pixels to which the contrast agent had access, showing a statistical significance for vₚ or Ktrans). FIG. 2A shows typical images in mice treated by BTTA or vehicle 3 days after intra-tumoral administration. The fraction of perfused pixels for the tumors treated with BTTA (FIG. 2B) was significantly greater than that of controls (69.2±3.4%, n=3 versus 39.9±4.7%, n=4 respectively, p<0.05, student t-test). No differences in the average values of K^{trans}, Kₑₚ or Vₚ were observed between tumors treated with BTTA or vehicle (FIG. 2, C-D). These results indicate that there are more areas of the tumor that are perfused after BTTA treatment with no changes in hemodynamic parameters. These results were further confirmed by a simple experiment in which a dye (Patent Blue) was administered by IV injection 1 minute before the sacrifice of the animal. After tumor excision and cutting two size-matched halves, the stained areas were digitally analyzed. FIG. 3 shows typical images and quantitative data: tumours treated with BTTA stained more positive (n=8, 61.7±3.8%) than control tumours (n=5, 30.2±4.4%). The difference was found to be statistically significant (unpaired t-test, p<0.01). These results demonstrate the better accessibility of the dye when the tumor was pre-treated by BTTA compared to controls. Finally, the Miles assay (FIG. 4) indicated that there was an increase in the uptake of albumin-Evans Blue complex by treated tumors (8.30±1.38 μg/g, n=4) compared to control tumors (4.38±0.92 μg/g, n=4). However, this increase was not statistically significant (p>0.05). These results are consistent with the DCE-MRI results (non significant increase of K^{trans}). The weak increase in permeability could be in part the result of the higher blood supply which may result in a higher EB uptake.

### Result 2

### BTTA Increases the Efficacy of Radiotherapy and Chemotherapy

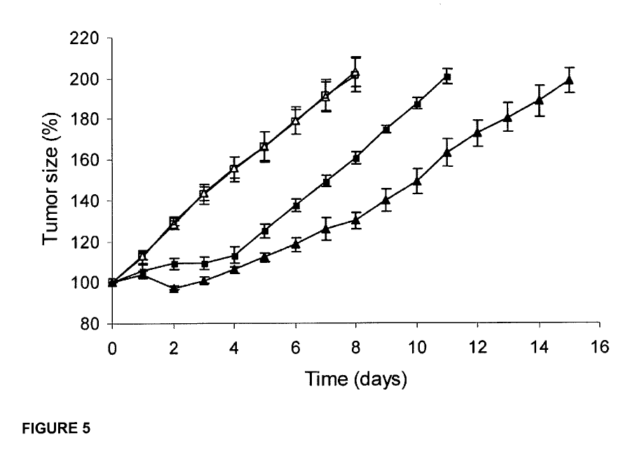

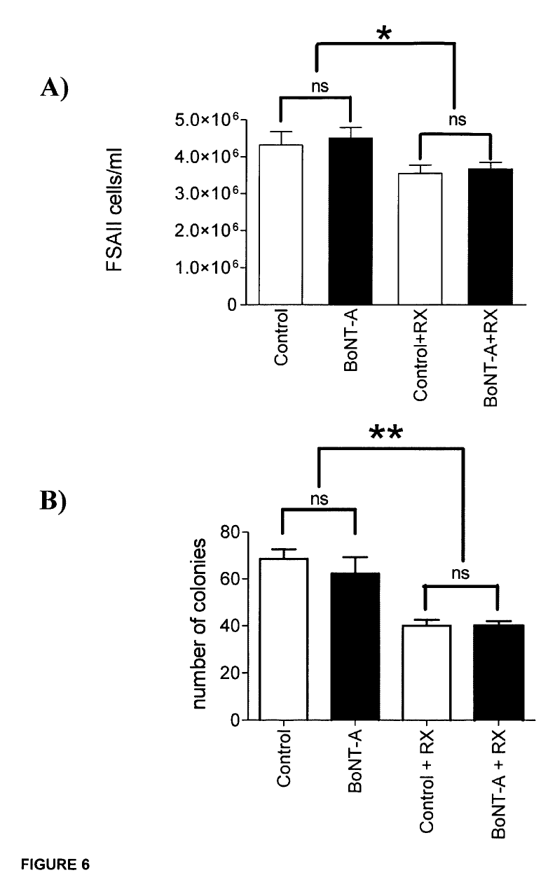

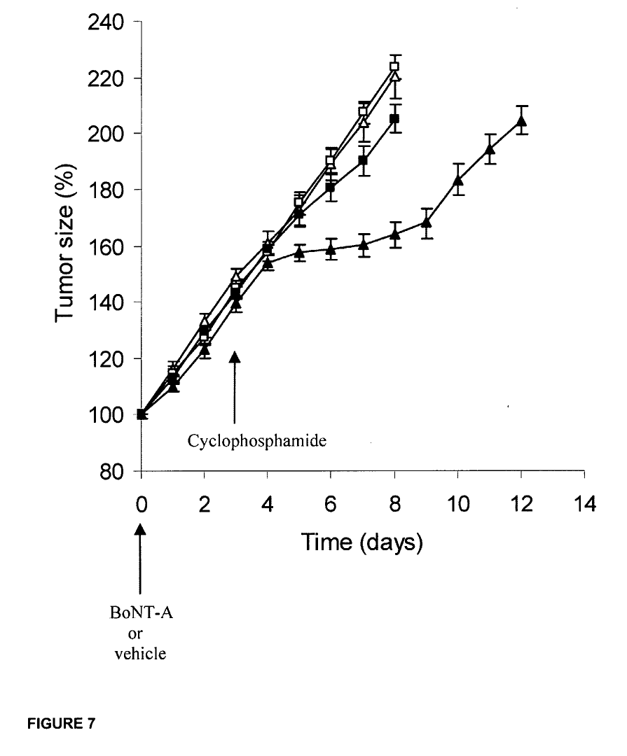

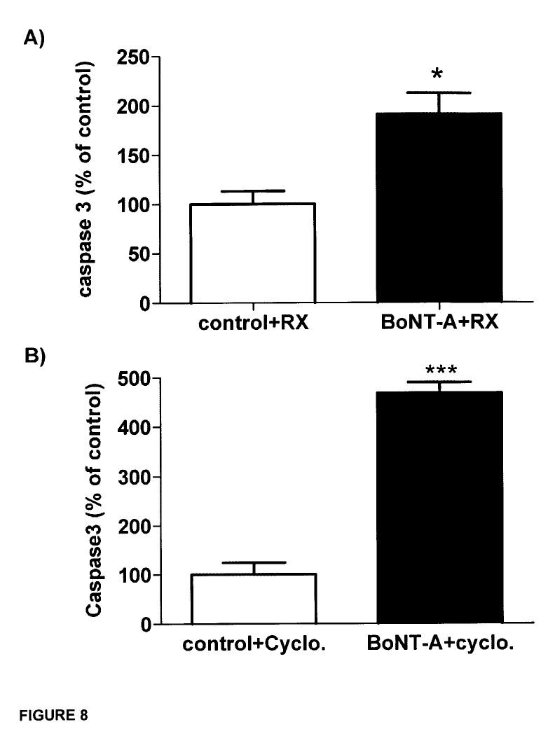

In order to assess the therapeutic relevance of the previous findings (significant increase in tumor oxygenation and perfusion), we investigated the potentiation of radiation therapy and chemotherapy by BTTA. FIG. 5 shows the tumor growth of FSaII tumors that receive injection of BTTA or vehicle, with or without irradiation at day 3 after administration (considered as day 0 on the irradiation graph). Interestingly, the administration of BTTA (n=6) did not affect the tumor growth, compared to the control group (n=5). When irradiated (without BTTA) with 20 Gy of RX, the tumor growth was delayed (regrowth delay for doubling the tumor size of 11.0±0.2 days, n=5). Pre-treatment with BTTA led to a significant increase in the tumor regrowth delay (15.7±1.0 days, n=6). To discriminate between an oxygen effect and a direct radiosensitizing effect, the radiosensitivity was tested on FSAII cells in the presence of BTTA (FIG. 6). Compared to control cells, BTTA did not exert any sensitizing effect regardless of whether a trypan blue exclusion dye method (FIG. 6 A) or clonogenic cell survival assay (FIG. 6 B) was used. Meanwhile, the 2-Gy irradiation led to a significant decrease for both experiments. These observations demonstrate that BTTA radiosensitizes tumors through changes in the tumor micro-environment rather than by a direct sensitizing effect. We next evaluated the possible potentiation of chemotherapy by BTTA. FIG. 7 shows the results from the experiment conducted on TLT tumors pretreated by BTTA or the vehicle, and receiving a suboptimal dose of cyclophosphamide (50 mg/kg) at day 3 after the treatment. In these conditions, the growth curves of the tumor did not differ while the tumor growth was significantly delayed in tumors receiving the combination BTTA and cyclophosphamide. The regrowth delay for doubling the tumor size was 7.7±0.4 days and 11.7±0.5 days for the groups receiving cyclophosphamide 3 days after administration of saline (n=7) and BTTA (n=6), respectively (significant difference, p<0.05, unpaired t-test). As expected, BTTA led to an increase in the apoptotic death associated with the radiotherapy and cyclophosphamide treatments. The activation of caspase-3 was 1.9 and 4.7 times increased when BTTA was associated with radiotherapy and chemotherapy, respectively (FIG. 8). Using BTTA alone (without radiotherapy or chemotherapy), we did not observe an activation of caspase-3 in these tumours. These results observed using caspase-3 were confirmed by an increase in the cleavage of PARP (data not shown).

### Result 3

### BTTA Interferes with the Tumor Vessels Neurogenic Contractions

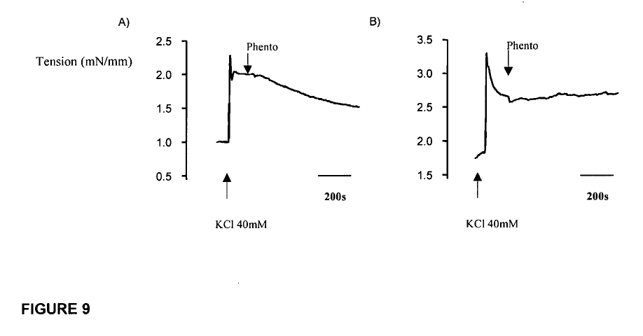

In striated muscles, BTTA inhibits the release of the neurotransmitter acetylcholine at the neuromuscular junction, thereby interfering with striated muscle contractile tone. Similarly, we hypothesized that BTTA could interfere with neurotransmitter release at the perivascular sympathetic varicosities, leading to inhibition of tumor vessel neurogenic contractions and therefore improvement of tumor perfusion and oxygenation. To test this hypothesis, we used a model of isolated arteries mounted in wire-myograph, which allowed us to monitor specifically the neurogenic tone developed by saphenous arterioles that were co-opted by the surrounding growing tumor cells. The vessels were challenged with a KCl solution in order to depolarize smooth muscle cells of the media and nerve endings, thereby also activating the Ca²⁺-dependent release of neurotransmitter. The amplitude of the neurotransmilter release was estimated by measuring the relaxation to an alpha-adrenoreceptor blocker (phentolamine) or to a cholinergic antagonist (atropine) for noradrenaline and acetycholine, respectively. We observed that in 4 vessels treated with BTTA, the relaxation to phentolamine was significantly smaller (52.4±12.8% of the control) confirming that BTTA could interfere with the release of neurotransmitters (e.g., noradrenaline) and neurogenic vasoconstriction (FIG. 9). A similar experimental protocol using atropine failed to reveal a neurogenic acetylcholine-evoked contraction.

### Example 2

All experiments were performed according to national animal care regulations. Transplantable Liver Tumors were implanted in the gastrocnemius muscle in the leg of 10 male NMRI mice (35-40 g, Animalerie facultaire, Catholic University of Leuven, Brussels). BTTA (Botox®, Allergan, Antwerp, Belgium, N=5) or saline solution (control, N=5) was injected when tumors reached a diameter of 8.0±1.0 mm. BTTA was administered directly into the tumor at two different sites (2 injections of 20 μl, corresponding to a total dose of 29 U kg⁻¹). In vivo nuclear magnetic resonance (NMR) proton and fluorine spectroscopy at 4.7 T (Bruker Biospec, Germany) were performed 2 days after BTTA or saline treatment. For these NMR experiments, gemcitabine (Gemzar), Eli Lilly, Belgium) was administered i.p. at a dose of 800 mg/kg and the animals were anesthetized with 1.5% isoflurane. A 25 mm diameter surface coil which could be tuned separately to either ¹H or ¹⁹F (Bruker, Germany) was placed directly over the tumor in such a way as to maximize the NMR signal received from the tumor and minimize the signal from the upper leg and paw. Non-localized proton (full spectrum, no water suppression) and fluorine spectroscopy were performed with the following parameters: a=90° (20-80 μs block RF pulse), spectral width=25 kHz, acq. size=8 k, TR=6 s, Navg=4 for proton, 150 for fluorine (total acq. time for the latter=15 min). Fluorine spectra were acquired approximately 40 minutes after the administration of gemcitabine. The free induction decay data were Fourier transformed (line broadening 25 Hz for fluorine spectra), phased, baseline corrected, and integrated (real part only, integration width 20 ppm for proton spectra, 12 ppm for gemcitabine peak of fluorine spectra) to obtain the gemcitabine and proton signals. Assuming uniform distribution of gemcitabine, unit density of tissue, and no NMR-invisible protons, the ratio of gemcitabine signal to proton signal provided a measure of the concentration of gemcitabine in mM (calibrated via separate phantom experiments). The precision of the gemcitabine concentration measurements was approximately 7% (˜0.15 mM).

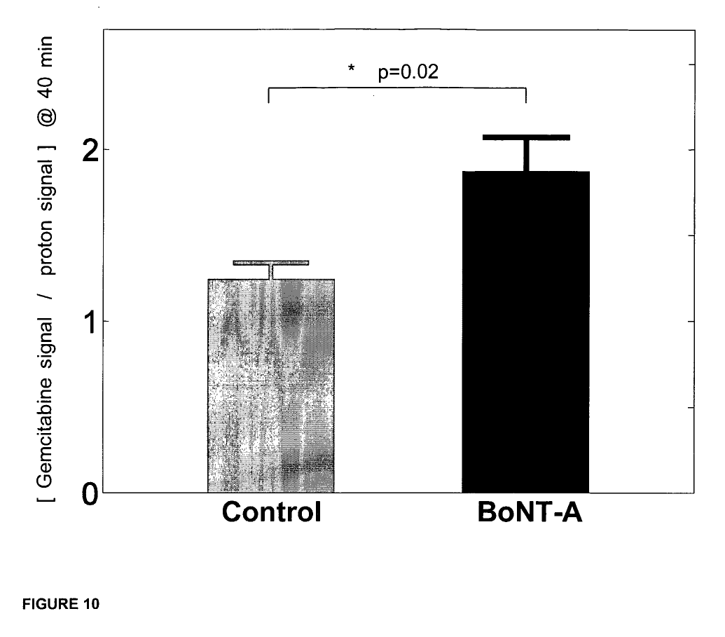

Results: The concentration of gemcitabine in tumors treated with BTTA was approximately 50% higher than in tumors treated with saline (p=0.02). The bar graph in FIG. 10 shows the concentration values for control and BTTA-treated tumors as mean±SE. The results show that Botulinum Toxin improves the delivery of the chemotherapy agent gemcitabine into experimental mouse tumors, as observed in vivo by NMR fluorine spectroscopy.

1. A method for enhancing cytotoxic therapy of cancerous cells or tumors comprising administering to said cells or tumors a pharmaceutical composition comprising at least one Botulinum toxin, BT.

2. The method according to claim 1, wherein said composition is administered locally to cancerous cells or tumors.

3. The method according to claim 1, wherein the cytotoxic therapy is radiotherapy, chemotherapy, systemic chemotherapy, or increasing the uptake of an active compound in the cancerous cells or tumors.

4. The method according to claim 3, wherein the cytotoxic therapy is radiotherapy and wherein the pharmaceutical composition is administered prior to radiotherapy.

5. The method according to claim 3, wherein the cytotoxic therapy is radiotherapy and wherein the pharmaceutical composition is administered during radiotherapy.

6-7. (canceled)

8. The method according to claim 3 wherein the cytotoxic therapy is chemotherapy or systemic chemotherapy and wherein a chemotherapy agent is administered simultaneously, separately or sequentially in respect of said pharmaceutical composition.

9. The method according to claim 3 wherein the cytotoxic therapy is chemotherapy or systemic chemotherapy and wherein said chemotherapy comprises administering a chemotherapy agent effective in causing the arrest or regression of cancerous cells or tumors in a subject.

10. The method according to claim 9, wherein said chemotherapy agent is any of taxol, gemcitabine or cis-platin or a combination thereof.

11. The method according to claim 1, wherein the BTs composition comprises two or more BTs which are administered simultaneously, separately or sequentially.

12. The method according to claim 1 wherein a pharmaceutical composition further comprises a suitable carrier material.

13. The method according to claim 1 wherein the cancer is any of gastric cancer, lung cancer, ovarian cancer, prostate cancer, liver cancer, uterine cancer, thyroid cancer, pancreatic cancer, lingual cancer, bile duct cancer, rectal cancer, mammary cancer, skin cancer, colon cancer, head and neck cancer or CNS cancer.

14. A pharmaceutical composition comprising at least one BT and at least one chemotherapy agent.

15. The pharmaceutical composition according to claim 14, for simultaneous, separate or sequential administration to a subject.

16. The pharmaceutical composition according to claim 14 wherein a polypeptide of a BT is replaced with a nucleic acid capable of encoding said polypeptide or a homologue or functional fragment thereof.

17. (canceled)

18. The pharmaceutical composition according to claim 14 wherein the at least one BT comprises a BT selected from the group consisting of Clostridium botulinum type A neurotoxin, BTTA; Clostridium botulinum type B neurotoxin, BTTB; Clostridium botulinum type C1 neurotoxin, BTTC1; Clostridium botulinum type C2 neurotoxin, BTTC2; Clostridium botulinum type C3 neurotoxin, BTTC3; Clostridium botulinum type D neurotoxin, BTTD: Clostridium botulinum type E neurotoxin, BTTE; Clostridium botulinum type F neurotoxin, BTTF; and Clostridium botulinum type G neurotoxin polypeptide, BTTG.

19. The pharmaceutical composition according to claim 18 wherein the BTTA comprises an active Clostridium botulinum type A neurotoxin polypeptide.

20. The pharmaceutical composition according to claim 18 wherein said BTTA comprises a homologue or functional fragment of an active Clostridium botulinum type A neurotoxin polypeptide.

21. The pharmaceutical composition according to claim 18 wherein the BTTA is Nc-224 or Nc-270.

22. (canceled)

23. The pharmaceutical composition according to claim 16 wherein said nucleic acid is selected from the group consisting of a nucleic acid sequence capable of encoding an active Clostridium botulinum type B neurotoxin polypeptide, a nucleic acid sequence capable of encoding an active Clostridium botulinum type A neurotoxin polypeptide, a nucleic acid sequence capable of encoding an active Clostridium botulinum type C1 neurotoxin polypeptide, a nucleic acid sequence capable of encoding an active BTTC2 component I or II polypeptide, a nucleic acid sequence capable of encoding an active BTTC3 polypeptide, a nucleic acid sequence capable of encoding an active BTTD polypeptide, a nucleic acid sequence capable of encoding an active BTTE polypeptide, a nucleic acid sequence capable of encoding an active BTTF polypeptide and a nucleic acid sequence capable of encoding an active BTTG polypeptide.

24. (canceled)

25. The pharmaceutical composition according to claim 18 wherein the BTTB comprises an active Clostridium botulinum type B neurotoxin polypeptide.

26. The pharmaceutical composition according to claim 18 wherein said BTTB comprises a homologue or functional fragment of an active Clostridium botulinum type B neurotoxin polypeptide.

27-29. (canceled)

30. The pharmaceutical composition according to claim 18 wherein the BTTC1 comprises an active Clostridium botulinum type C1 neurotoxin polypeptide.

31. The pharmaceutical composition according to claim 18 wherein said BTTC1 comprises a homologue or functional fragment of an active Clostridium botulinum type C1 neurotoxin polypeptide.

32-33. (canceled)

34. The pharmaceutical composition according to claim 18 wherein said BTTC2, comprises BTTC2 component I.

35. The pharmaceutical composition according to claim 18 wherein said BTTC2, comprises BTTC2 component II.

36. The pharmaceutical composition according to claim 34 wherein the BTTC2 component I comprises an active Clostridium botulinum type C2 neurotoxin component I polypeptide.

37. The pharmaceutical composition according to claim 34 wherein said BTTC2 component I comprises a homologue or functional fragment of an active Clostridium botulinum type C2 neurotoxin component I polypeptide.

38. The pharmaceutical composition according to claim 35 wherein the BTTC2 component II comprises an active Clostridium botulinum type C2 neurotoxin component II polypeptide.

39. The pharmaceutical composition according to claim 35 wherein said BTTC2 component II comprises a homologue or functional fragment of an active Clostridium botulinum type C2 neurotoxin component II polypeptide.

40-41. (canceled)

42. The pharmaceutical composition according to claim 18 wherein the BTTC3 comprises an active Clostridium botulinum type C3 neurotoxin polypeptide.

43. The pharmaceutical composition according to claim 18 wherein said BTTC3 comprises a homologue or functional fragment of an active Clostridium botulinum type C3 neurotoxin polypeptide.

44-45. (canceled)

46. The pharmaceutical composition according to claim 18 wherein the BTTD comprises an active Clostridium botulinum type D neurotoxin polypeptide.

47. The pharmaceutical composition according to claim 18 wherein said BTTD comprises a homologue or functional fragment of an active Clostridium botulinum type D neurotoxin polypeptide.

48-49. (canceled)

50. The pharmaceutical composition according to claim 18 wherein the BTTE comprises an active Clostridium botulinum type E neurotoxin polypeptide.

51. The pharmaceutical composition according to claim 18 wherein said BTTE comprises a homologue or functional fragment of an active Clostridium botulinum type E neurotoxin polypeptide.

52-53. (canceled)

54. The pharmaceutical composition according to claim 18 wherein the BTTF comprises an active Clostridium botulinum type F neurotoxin polypeptide.

55. The pharmaceutical composition according to claim 18 wherein said BTTF comprises a homologue or functional fragment of an active Clostridium botulinum type F neurotoxin polypeptide.

56-57. (canceled)

58. The pharmaceutical composition according to claim 18 wherein the BTTG comprises an active Clostridium botulinum type G neurotoxin polypeptide.

59. The pharmaceutical composition according to claim 18 wherein said BTTG comprises a homologue or functional fragment of an active Clostridium botulinum type G neurotoxin polypeptide.

60-77. (canceled)

78. The method according to claim 1, wherein a polypeptide of BT is replaced with a nucleic acid capable of encoding said polypeptide.

79. The method according to claim 78, wherein said nucleic acid is capable of encoding a homologue or functional fragment of said BT.

80. (canceled)

81. The method according to claim 1, wherein the at least one BT comprises a BT selected from the group consisting of Clostridium botulinum type A neurotoxin, BTTA; Clostridium botulinum type B neurotoxin, BTTB; Clostridium botulinum type C1 neurotoxin, BTTC1; Clostridium botulinum type C2 neurotoxin, BTTC2; Clostridium botulinum type C3 neurotoxin, BTTC3; Clostridium botulinum type D neurotoxin, BTTD; Clostridium botulinum type E neurotoxin, BTTE; Clostridium botulinum type F neurotoxin, BTTF; and Clostridium botulinum type G neurotoxin polypeptide, BTTG.
